SEQUENCE LISTING (20 sequences)

SEQ ID NO: 1 - DNA, 2811 bp, Corynebacterium glutamicum
  CDS (373)..(2022): pgi
aaaacccgaggggcgaaaattccaccctaacttttttgggatcccctttttccggggaattaattggtttgggtttcaatgggaaaacgggaaacaatgggccaaaggttcaaaaaccccaaaagggggccgggttcaaattcccaaaaaaaatggcaaaaaagggggggccaaaaccaagttggcccccaaaccaccggggcaacggcccacccacaaaggggttgggttaaaggaaggacgcccaaagtaagcccggaatggcccacgttcgaaaaagcaggccccaattaaacgcaccttaaatttgtcgtgtttcccactttgaacactcttcgatgcgcttggccacaaaagcaagctaacctgaagatgttatttaacgacaataaaggagttttcatggcggacatttcgaccacccaggtttggcaagacctgaccgatcattactcaaacttccaggcaaccactctgcgtgaacttttcaaggaagaaaaccgcgccgagaagtacaccttctccgcggctggcctccacgtcgacctgtcgaagaatctgcttgacgacgccaccctcaccaagctccttgcactgaccgaagaatctggccttcgcgaacgcattgacgcgatgtttgccggtgaacacctcaacaacaccgaagaccgcgctgtcctccacaccgcgctgcgccttcctgccgaagctgatctgtcagtagatggccaagatgttgctgctgatgtccacgaagttttgggacgcatgcgtgacttcgctactgcgctgcgctcaggcaactggttgggacacaccggccacacgatcaagaagatcgtcaacattggtatcggtggctctgacctcggaccagccatggctacgaaggctctgcgtgcatacgcgaccgctggtatctcagcagaattcgtctccaacgtcgacccagcagacctcgtttctgtgttggaagacctcgatgcagaatccacattgttcgtgatcgcttcgaaaactttcaccacccaggagacgctgtccaacgctcgtgcagctcgtgcttggctggtagagaagctcggtgaagaggctgtcgcgaagcacttcgtcgcagtgtccaccaatgctgaaaaggtcgcagagttcggtatcgacacggacaacatgttcggcttctgggactgggtcggaggtcgttactccgtggactccgcagttggtctttccctcatggcagtgatcggccctcgcgacttcatgcgtttcctcggtggattccacgcgatggatgaacacttccgcaccaccaagttcgaagagaacgttccaatcttgatggctctgctcggtgtctggtactccgatttctatggtgcagaaacccacgctgtcctaccttattccgaggatctcagccgttttgctgcttacctccagcagctgaccatggagaccaatggcaagtcagtccaccgcgacggctcccctgtttccactggcactggcgaaatttactggggtgagcctggcacaaatggccagcacgctttcttccagctgatccaccagggcactcgccttgttccagctgatttcattggtttcgctcgtccaaagcaggatcttcctgccggtgagcgcaccatgcatgaccttttgatgagcaacttcttcgcacagaccaaggttttggctttcggtaagaacgctgaagagatcgctgcggaaggtgtcgcacctgagctggtcaaccacaaggtcgtgccaggtaatcgcccaaccaccaccattttggcggaggaacttaccccttctattctcggtgcgttgatcgctttgtacgaacacaccgtgatggttcagggcgtgatttgggacatcaactccttcgaccaatggggtgttgaactgggcaaacagcaggcaaatgacctcgctccggctgtctctggtgaagaggatgttgactcgggagattcttccactgattcactgattaagtggtaccgcgcaaataggtagtcgcttgcttatagggtcaggggcgtgaagaatcctcgcctcatagcactggccgctatcatcctgacctcgttcaatctgcgaacagctattactgctttagctccgctggtttctgagattcgggatgatttaggggttagtgcttctcttattggtgtgttgggcatgatcccgactgctatgttcgcggttgctgcgtttgcgcttccgtcgttgaagaggaagttcactacttcccaactgttgatgtttgccatgctgttgactgctgccggtcagattattcgtgtcgctggacctgcttcgctgttgatggtcggtactgtgttcgcgatgtttgcgatcggagttaccaatgtgttgcttccgattgctgttagggagtattttccgcgtcacgtcggtggaatgtcgacaacttatctggtgtcgttccagattgttcaggcacttgctccgacgcttgccgtgccgatttctcagtgggctacacatgtggggttgaccggttggagggtgtcgctcggttcgtgggcgctgctggggttggttgcggcgatttcgtggattccgctgttgagtttgcagggtgccagggttgttgcggcgccgtcgaaggtttctcttcctgtgtggaagtcttcggttggtgtggggctcgggttgatgtttgggtttacttcgtttgcgacgtatatcctcatgggttttatgccgcagatggtaggtgatccaaagaattcaaaaagcttctcgagagtacttctagagcggccgcgggcc

SEQ ID NO: 2 - protein, 550 aa, Corynebacterium glutamicum
MLFNDNKGVFMADISTTQVWQDLTDHYSNFQATTLRELFKEENRAEKYTFSAAGLHVDLSKNLLDDATLTKLLALTEESGLRERIDAMFAGEHLNNTEDRAVLHTALRLPAEADLSVDGQDVAADVHEVLGRMRDFATALRSGNWLGHTGHTIKKIVNIGIGGSDLGPAMATKALRAYATAGISAEFVSNVDPADLVSVLEDLDAESTLFVIASKTFTTQETLSNARAARAWLVEKLGEEAVAKHFVAVSTNAEKVAEFGIDTDNMFGFWDWVGGRYSVDSAVGLSLMAVIGPRDFMRFLGGFHAMDEHFRTTKFEENVPILMALLGVWYSDFYGAETHAVLPYSEDLSRFAAYLQQLTMETNGKSVHRDGSPVSTGTGEIYWGEPGTNGQHAFFQLIHQGTRLVPADFIGFARPKQDLPAGERTMHDLLMSNFFAQTKVLAFGKNAEEIAAEGVAPELVNHKVVPGNRPTTTILAEELTPSILGALIALYEHTVMVQGVIWDINSFDQWGVELGKQQANDLAPAVSGEEDVDSGDSSTDSLIKWYRANR

SEQ ID NO: 3 - DNA, 462 bp, Corynebacterium glutamicum
atggagaccaatggcaagtcagtccaccgcgacggctcccctgtttccactggcactggcgaaatttactggggtgagcctggcacaaatggccagcacgctttcttccagctgatccaccagggcactcgccttgttccagctgatttcattggtttcgctcgtccaaagcaggatcttcctgccggtgagcgcaccatgcatgaccttttgatgagcaacttcttcgcacagaccaaggttttggctttcggtaagaacgctgaagagatcgctgcggaaggtgtcgcacctgagctggtcaaccacaaggtcgtgccaggtaatcgcccaaccaccaccattttggcggaggaacttaccccttctattctcggtgcgttgatcgctttgtacgaacacaccgtgatggttcagggcgtgatttgggacatcaactccttcgaccaatggggcgtggaa

SEQ ID NO: 4 - DNA, 2160 bp, Corynebacterium glutamicum
  CDS (327)..(2063): poxB
ttagaggcgattctgtgaggtcactttttgtggggtcggggtctaaatttggccagttttcgaggcgaccagacaggcgtgcccacgatgtttaaataggcgatcggtgggcatctgtgtttggtttcgacgggctgaaaccaaaccagactgcccagcaacgacggaaatcccaaaagtgggcatccctgtttggtaccgagtacccacccgggcctgaaactccctggcaggcgggcgaagcgtggcaacaactggaatttaagagcacaattgaagtcgcaccaagttaggcaacacaatagccataacgttgaggagttcagatggcacacagctacgcagaacaattaattgacactttggaagctcaaggtgtgaagcgaatttatggtttggtgggtgacagccttaatccgatcgtggatgctgtccgccaatcagatattgagtgggtgcacgttcgaaatgaggaagcggcggcgtttgcagccggtgcggaatcgttgatcactggggagctggcagtatgtgctgcttcttgtggtcctggaaacacacacctgattcagggtctttatgattcgcatcgaaatggtgcgaaggtgttggccatcgctagccatattccgagtgcccagattggttcgacgttcttccaggaaacgcatccggagattttgtttaaggaatgctctggttactgcgagatggtgaatggtggtgagcagggtgaacgcattttgcatcacgcgattcagtccaccatggcgggtaaaggtgtgtcggtggtagtgattcctggtgatatcgctaaggaagacgcaggtgacggtacttattccaattccactatttcttctggcactcctgtggtgttcccggatcctactgaggctgcagcgctggtggaggcgattaacaacgctaagtctgtcactttgttctgcggtgcgggcgtgaagaatgctcgcgcgcaggtgttggagttggcggagaagattaaatcaccgatcgggcatgcgctgggtggtaagcagtacatccagcatgagaatccgtttgaggtcggcatgtctggcctgcttggttacggcgcctgcgtggatgcgtccaatgaggcggatctgctgattctattgggtacggatttcccttattctgatttccttcctaaagacaacgttgcccaggtggatatcaacggtgcgcacattggtcgacgtaccacggtgaagtatccggtgaccggtgatgttgctgcaacaatcgaaaatattttgcctcatgtgaaggaaaaaacagatcgttccttccttgatcggatgctcaaggcacacgagcgtaagttgagctcggtggtagagacgtacacacataacgtcgagaagcatgtgcctattcaccctgaatacgttgcctctattttgaacgagctggcggataaggatgcggtgtttactgtggataccggcatgtgcaatgtgtggcatgcgaggtacatcgagaatccggagggaacgcgcgactttgtgggttcattccgccacggcacgatggctaatgcgttgcctcatgcgattggtgcgcaaagtgttgatcgaaaccgccaggtgatcgcgatgtgtggcgatggtggtttgggcatgctgctgggtgagcttctgaccgttaagctgcaccaacttccgctgaaggctgtggtgtttaacaacagttctttgggcatggtgaagttggagatgctcgtggagggacagccagaatttggtactgaccatgaggaagtgaatttcgcagagattgcggcggctgcgggtatcaaatcggtacgcatcaccgatccgaagaaagttcgcgagcagctagctgaggcattggcatatcctggacctgtactgatcgatatcgtcacggatcctaatgcgctgtcgatcccaccaaccatcacgtgggaacaggtcatgggattcagcaaggcggccacccgaaccgtctttggtggaggagtaggagcgatgatcgatctggcccgttcgaacataaggaatattcctactccatgatgattgatacacctgctgttctcattgaccgcgagcgcttaactgccaacatttccaggatggcagctcacgccggtgcccatgagattgccct

SEQ ID NO: 5 - protein, 579 aa, Corynebacterium glutamicum
MAHSYAEQLIDTLEAQGVKRIYGLVGDSLNPIVDAVRQSDIEWVHVRNEEAAAFAAGAESLITGELAVCAASCGPGNTHLIQGLYDSHRNGAKVLAIASHIPSAQIGSTFFQETHPEILFKECSGYCEMVNGGEQGERILHHAIQSTMAGKGVSVVVIPGDIAKEDAGDGTYSNSTISSGTPVVFPDPTEAAALVEAINNAKSVTLFCGAGVKNARAQVLELAEKIKSPIGHALGGKQYIQHENPFEVGMSGLLGYGACVDASNEADLLILLGTDFPYSDFLPKDNVAQVDINGAHIGRRTTVKYPVTGDVAATIENILPHVKEKTDRSFLDRMLKAHERKLSSVVETYTHNVEKHVPIHPEYVASILNELADKDAVFTVDTGMCNVWHARYIENPEGTRDFVGSFRHGTMANALPHAIGAQSVDRNRQVIAMCGDGGLGMLLGELLTVKLHQLPLKAVVFNNSSLGMVKLEMLVEGQPEFGTDHEEVNFAEIAAAAGIKSVRITDPKKVREQLAEALAYPGPVLIDIVTDPNALSIPPTITWEQVMGFSKAATRTVFGGGVGAMIDLARSNIRNIPTP

SEQ ID NO: 6 - DNA, 875 bp, Corynebacterium glutamicum
tgcgagatggtgaatggtggtgagcagggtgaacgcattttgcatcacgcgattcagtccaccatggcgggtaaaggtgtgtcggtggtagtgattcctggtgatatcgctaaggaagacgcaggtgacggtacttattccaattccactatttcttctggcactcctgtggtgttcccggatcctactgaggctgcagcgctggtggaggcgattaacaacgctaagtctgtcactttgttctgcggtgcgggcgtgaagaatgctcgcgcgcaggtgttggagttggcggagaagattaaatcaccgatcgggcatgcgctgggtggtaagcagtacatccagcatgagaatccgtttgaggtcggcatgtctggcctgcttggttacggcgcctgcgtggatgcgtccaatgaggcggatctgctgattctattgggtacggatttcccttattctgatttccttcctaaagacaacgttgcccaggtggatatcaacggtgcgcacattggtcgacgtaccacggtgaagtatccggtgaccggtgatgttgctgcaacaatcgaaaatattttgcctcatgtgaaggaaaaaacagatcgttccttccttgatcggatgctcaaggcacacgagcgtaagttgagctcggtggtagagacgtacacacataacgtcgagaagcatgtgcctattcaccctgaatacgttgcctctattttgaacgagctggcggataaggatgcggtgtttactgtggataccggcatgtgcaatgtgtggcatgcgaggtacatcgagaatccggagggaacgcgcgactttgtgggttcattccgccacggcacgatggctaatgcgttgcctcatgc

SEQ ID NO: 7 - DNA, 2260 bp, Brevibacterium flavum MJ-233
  CDS (629)..(2080): Glucose-6-Phosphate Dehydrogenase (EC 1.1.1.49); JP-A-09-22461
gatccgatgaggctttggctctgcgtggcaaggcaggcgttgccaacgctcagcgcgcttacgctgtgtacaaggagcttttcgacgccgccgagctgcctgtaaggcgccaacactcagcgcccactgtgggcatccaccggcgtgaagaaccctgcgtacgctgcaactctttacgtttccgagctggctggtccaaacaccgtcaacaccatgccagaaggcaccatcgacgctgttctggaactgggcaacctgcacggtgacaacctgtccaactccgcggcagaagctgacgctgtgttctcccagcttgaggctctgggcgttgacttggcagatgtcttccaggtcctggagaccgaggccgtggacaagttcgttgcttcttggagcgaactgcttgagtccatggaagctcgcctgaagtagaatcagcacgctgcatcagtaacggcgacatgaaatcgaattagttcgatcttatgtggccgttacacatctttcattaaagaaaggatcgtgacgcttaccatcgtgagcacaaaacacgaccccctccagctggacaaacccactgcgcgacccgcaggataaacgactcccccgcatcgctggcccttccggcatggtgatcttcggtgtcactggcgacttggctcgaaagaagctgctccccgccatttatgatctagcaaaccgcggattgctgcccccaggattctcgttggtaggttacggccgccgcgaatggtccaaagaagactttgaaaaatacgtacgcgatgccgcaagtgctggtgctcgtacggaattccgtgaaaatgtttgggagcgcctcgccgagggtatggaatttgttcgcggcaactttgatgatgatgcagctttcgacaacctcgctgcaacactcaagcgcatcgacaaaacccgcggcaccgccggcaactgggcttactacctgtccattccaccagattccttcgcagcggtctgccaccagctggagcgttccggcatggctgaatccaccgaagaagcatggcgccgcgtgatcatcgagaagcctttcggccacaacctcgaatccgcacacgagctcaaccagctggtcaacgcagtcttcccagaatcttctgtgttccgcatcgaccactatttgggcaaggaaacagttcaaaacatcctggctctgcgttttgctaaccagctgtttgagccactgtggaactccaactacgttgaccacgtccagatcaccatggctgaagatattggcttgggtggacgtgctggttactacgacggcatcggcgcagcccgcgacgtcatccagaaccacctgatccagctcttggctctggttgccatggaagaaccaatttctttcgtgccagcgcagctgcaggcagaaaagatcaaggtgctctctgcgacaaagccgtgctacccattggataaaacctccgctcgtggtcagtacgctgccggttggcagggctctgagttagtcaagggacttcgcgaagaagatggcttcaaccctgagtccaccactgagacttttgcggcttgtaccttagagatcacgtctcgtcgctgggctggtgtgccgttctacctgcgcaccggtaagcgtcttggtcgccgtgttactgagattgccgtggtgtttaaagacgcaccacaccagcctttcgacggcgacatgactgtatcccttggccaaaacgccatcgtgattcgcgtgcagcctgatgaaggtgtgctcatccgcttcggttccaaggttccaggttctgccatggaagtccgtgacgtcaacatggacttctcctactcagaatccttcactgaagaatcacctgaagcatacgagcgccttatcttggatgcgctgttggatgaatccagccttttccctaccaacgaggaagtggaactgagctggaagattctggatccaattcttgaagcatgggatgccgatggagaaccagaggattacccagcaggtacgtggggtccaaagagcgctgatgaaatgctttcccgcaacggtcacacctggcgcaggccataatttaggggcaaaaaatgatctttgaacttccggataccaccacccagcaaatttccaagaccctaactcgactgcgtgaatcgggcacccaggtcaccaccggccgagtgctcaccctcatcgtggtcactgactccgaaagcgatgtcgctgcagttaccgagtccaccaatgaag

SEQ ID NO: 8 - protein, 484 aa, Brevibacterium flavum MJ-233
MVIFGVTGDLARKKLLPAIYDLANRGLLPPGFSLVGYGRREWSKEDFEKYVRDAASAGARTEFRENVWERLAEGMEFVRGNFDDDAAFDNLAATLKRIDKTRGTAGNWAYYLSIPPDSFAAVCHQLERSGMAESTEEAWRRVIIEKPFGHNLESAHELNQLVNAVFPESSVFRIDHYLGKETVQNILALRFANQLFEPLWNSNYVDHVQITMAEDIGLGGRAGYYDGIGAARDVIQNHLIQLLALVAMEEPISFVPAQLQAEKIKVLSATKPCYPLDKTSARGQYAAGWQGSELVKGLREEDGFNPESTTETFAACTLEITSRRWAGVPFYLRTGKRLGRRVTEIAVVFKDAPHQPFDGDMTVSLGQNAIVIRVQPDEGVLIRFGSKVPGSAMEVRDVNMDFSYSESFTEESPEAYERLILDALLDESSLFPTNEEVELSWKILDPILEAWDADGEPEDYPAGTWGPKSADEMLSRNGHTWRRP

SEQ ID NO: 9 - DNA, 2259 bp, Corynebacterium glutamicum
  CDS (538)..(2079): Zwf-Protein
gatccgatgaggctttggctctgcgtggcaaggcaggcgttgccaacgctcagcgcgcttacgctgtgtacaaggagcttttcgacgccgccgagctgcctgtaaggcgccaacactcagcgcccactgtgggcatccaccggcgtgaagaaccctgcgtacgctgcaactctttacgtttccgagctggctggtccaaacaccgtcaacaccatgccagaaggcaccatcgacgctgttctggaactgggcaacctgcacggtgacaacctgtccaactccgcggcagaagctgacgctgtgttctcccagcttgaggctctgggcgttgacttggcagatgtcttccaggtcctggagaccgaggccgtggacaagttcgttgcttcttggagcgaactgcttgagtccatggaagctcgcctgaagtagaatcagcacgctgcatcagtaacggcgacatgaaatcgaattagttcgatcttatgtggccgttacacatctttcattaaagaaaggatcgtgacgcttaccatcgtgagcacaaacacgaccccctccagctggacaaacccactgcgcgacccgcaggataaacgactcccccgcatcgctggcccttccggcatggtgatcttcggtgtcactggcgacttggctcgaaagaagctgctccccgccatttatgatctagcaaaccgcggattgctgcccccaggattctcgttggtaggttacggccgccgcgaatggtccaaagaagactttgaaaaatacgtacgcgatgccgcaagtgctggtgctcgtacggaattccgtgaaaatgtttgggagcgcctcgccgagggtatggaatttgttcgcggcaactttgatgatgatgcagctttcgacaacctcgctgcaacactcaagcgcatcgacaaaacccgcggcaccgccggcaactgggcttactacctgtccattccaccagattccttcgcagcggtctgccaccagctggagcgttccggcatggctgaatccaccgaagaagcatggcgccgcgtgatcatcgagaagcctttcggccacaacctcgaatccgcacacgagctcaaccagctggtcaacgcagtcttcccagaatcttctgtgttccgcatcgaccactatttgggcaaggaaacagttcaaaacatcctggctctgcgttttgctaaccagctgtttgagccactgtggaactccaactacgttgaccacgtccagatcaccatggctgaagatattggcttgggtggacgtgctggttactacgacggcatcggcgcagcccgcgacgtcatccagaaccacctgatccagctcttggctctggttgccatggaagaaccaatttctttcgtgccagcgcagctgcaggcagaaaagatcaaggtgctctctgcgacaaagccgtgctacccattggataaaacctccgctcgtggtcagtacgctgccggttggcagggctctgagttagtcaagggacttcgcgaagaagatggcttcaaccctgagtccaccactgagacttttgcggcttgtaccttagagatcacgtctcgtcgctgggctggtgtgccgttctacctgcgcaccggtaagcgtcttggtcgccgtgttactgagattgccgtggtgtttaaagacgcaccacaccagcctttcgacggcgacatgactgtatcccttggccaaaacgccatcgtgattcgcgtgcagcctgatgaaggtgtgctcatccgcttcggttccaaggttccaggttctgccatggaagtccgtgacgtcaacatggacttctcctactcagaatccttcactgaagaatcacctgaagcatacgagcgccttatcttggatgcgctgttggatgaatccagccttttccctaccaacgaggaagtggaactgagctggaagattctggatccaattcttgaagcatgggatgccgatggagaaccagaggattacccagcaggtacgtggggtccaaagagcgctgatgaaatgctttcccgcaacggtcacacctggcgcaggccataatttaggggcaaaaaatgatctttgaacttccggataccaccacccagcaaatttccaagaccctaactcgactgcgtgaatcgggcacccaggtcaccaccggccgagtgctcaccctcatcgtggtcactgactccgaaagcgatgtcgctgcagttaccgagtccaccaatgaag

SEQ ID NO: 10 - protein, 514 aa, Corynebacterium glutamicum
MSTNTTPSSWTNPLRDPQDKRLPRIAGPSGMVIFGVTGDLARKKLLPAIYDLANRGLLPPGFSLVGYGRREWSKEDFEKYVRDAASAGARTEFRENVWERLAEGMEFVRGNFDDDAAFDNLAATLKRIDKTRGTAGNWAYYLSIPPDSFAAVCHQLERSGMAESTEEAWRRVIIEKPFGHNLESAHELNQLVNAVFPESSVFRIDHYLGKETVQNILALRFANQLFEPLWNSNYVDHVQITMAEDIGLGGRAGYYDGIGAARDVIQNHLIQLLALVAMEEPISFVPAQLQAEKIKVLSATKPCYPLDKTSARGQYAAGWQGSELVKGLREEDGFNPESTTETFAACTLEITSRRWAGVPFYLRTGKRLGRRVTEIAVVFKDAPHQPFDGDMTVSLGQNAIVIRVQPDEGVLIRFGSKVPGSAMEVRDVNMDFSYSESFTEESPEAYERLILDALLDESSLFPTNEEVELSWKILDPILEAWDADGEPEDYPAGTWGPKSADEMLSRNGHTWRRP

SEQ ID NO: 11 - DNA, 20 bp, Artificial Sequence
  Description of Artificial Sequence Primer zwf-forward
tcgacgcggttctggagcag

SEQ ID NO: 12 - DNA, 21 bp, Artificial Sequence
  Description of Artificial Sequence Primer zwf-reverse
ctaaattatggcctgcgccag

SEQ ID NO: 13 - DNA, 22 bp, Artificial Sequence
  Description of Artificial Sequence Universal forward Primer
gtaatacgactcactatagggc

SEQ ID NO: 14 - DNA, 18 bp, Artificial Sequence
  Description of Artificial Sequence M13 reverse primer
ytccacgccccaytgrtc

SEQ ID NO: 15 - DNA, 18 bp, Artificial Sequence
  Description of Artificial Sequence Internal Primer 1
ggaaacaggggagccgtc

SEQ ID NO: 16 - DNA, 18 bp, Artificial Sequence
  Description of Artificial Sequence Internal Primer 2
tgctgagataccagcggt

SEQ ID NO: 17 - DNA, 17 bp, Artificial Sequence
  Description of Artificial Sequence fwd primer
atggarwccaaygghaa

SEQ ID NO: 18 - DNA, 18 bp, Artificial Sequence
  Description of Artificial Sequence rev. primer
ytccacgccccaytgrtc

SEQ ID NO: 19 - DNA, 20 bp, Artificial Sequence
  Description of Artificial Sequence Primer poxBint1
tgcgagatggtgaatggtgg

SEQ ID NO: 20 - DNA, 20 bp, Artificial Sequence
  Description of Artificial Sequence Primer poxBint2
gcatgaggcaacgcattagc

(19) United States
(12) Patent Application Publication
(10) Pub. No.: US 2005/0112733 A1
(43) Pub. Date: May 26, 2005

(54) Process for the preparation of L-amino acids with amplification of the zwf gene

(75) Inventors: Kevin Burke, Galway (IE); Hermann Sahm, Julich (DE); Lothar Eggeling, Julich (DE); Bernd Moritz, Niederzier (DE); L. K. Dunican, Galway (IE); Ashling McCormack, Westmeath (IE); Cliona Stapleton, Roscrea (IE); Bettina Mockel, Bielefeld (DE); Georg Thierbach, Bielefeld (DE); Rita Dunican, Galway (IE)

Correspondence Address: FITCH, EVEN, TABIN & FLANNERY, P. O. BOX 65973, WASHINGTON, DC, 20035, US

(73) Assignee: Degussa AG

(21) Appl. No.: 11/025,115

(22) Filed: Dec. 30, 2004

Related U.S. Application Data
(63) Continuation of application No. 10/091,342, filed on Mar. 6, 2002.
Application No. 10/091,342 is a continuation-in-part of application No. 09/531,269, filed on Mar. 20, 2000, now abandoned.

(51) Int. Cl. C12P 13/08; C12N 1/21; C12N 15/74

(52) U.S. Cl. 435/115; 435/320.1; 435/252.3; 536/23.2

(57) ABSTRACT

The invention relates to a process for the preparation of L-amino acids. The process involves fermenting an L-amino acid producing coryneform bacteria in a culture medium, concentrating L-amino acid in the culture medium or in the cells of the bacteria, and isolating the L-amino acid produced. The bacteria has an amplified gene encoding the Zwischenferment protein.

### CROSS-REFERENCE TO RELATED APPLICATIONS

This is a continuation-in-part of U.S. application Ser. No. 09/531,269, filed Mar. 20, 2000, the contents of which are incorporated by reference herein in their entirety.

### FIELD OF THE INVENTION

The invention relates to a process for the preparation of L-amino acids, in particular L-lysine, L-threonine and L-tryptophan, using coryneform bacteria in which at least the Zwischenferment protein encoded by the zwf gene is amplified.

### DESCRIPTION OF BACKGROUND ART

L-Amino acids are used in animal nutrition, in human medicine and in the pharmaceuticals industry. It is known that amino acids are prepared by fermentation of strains of coryneform bacteria, in particular Corynebacterium glutamicum. Because of its great importance, work is constantly being undertaken to improve the preparation process. Improvements to the process can relate to fermentation measures, such as e.g. stirring and supply of oxygen, or the composition of the nutrient media, such as e.g. the sugar concentration during the fermentation, or the working up to the product form by e.g. ion exchange chromatography, or the intrinsic output properties of the microorganism itself.

Methods of mutagenesis, selection and mutant selection are used to improve the output properties of these microorganisms. Strains which are resistant to antimetabolites, such as e.g. the threonine analogue α-amino-β-hydroxyvaleric acid (AHV), the lysine analogue S-(2-aminoethyl)-L-cystein (AEC), or are auxotrophic for metabolites of regulatory importance and produce L-amino acids such as e.g. threonine or lysine are obtained in this manner.

Methods of the recombinant DNA technique have also been employed for some years for improving the strain of Corynebacterium glutamicum strains which produce L-amino acids.

### SUMMARY OF THE INVENTION

L-Amino acids are used in human medicine and in the pharmaceuticals industry, in the foodstuffs industry and especially in animal nutrition. There is therefore a general interest in providing new improved processes for the preparation of amino acids.

In general, the embodiments of the present invention provide new improved processes for the fermentative preparation of L-amino acids with coryneform bacteria. More specifically, the embodiments of the invention provide a process for the preparation of L-amino acids, in particular L-lysine, L-threonine, L-isoleucine and L-tryptophan, using coryneform bacteria in which the Zwischenferment protein (Zwf protein) encoded by the nucleotide sequence of the zwf gene is amplified, in particular over-expressed. The abbreviation “zwf” is a mnemonic for “Zwischenferment” (Jeffrey H. Miller: A Short Course In Bacterial Genetics, Cold Spring Harbor Laboratory Press, USA, 1992) and also referred to as glucose 6-phosphate dehydrogenase.

### BRIEF DESCRIPTION OF THE DRAWINGS

Embodiments of the invention will be described with reference to the following Figures, in which the base pair numbers stated are approximate values obtained in the context of reproducibility, and in which:

FIG. 1 is a map of the plasmid pEC-T18mob2;

FIG. 2 is a map of the plasmid pEC-T18mob2zwf;

FIG. 3 is a map of the plasmid pAMC1;

FIG. 4 is a map of the plasmid pMC1; and

FIG. 5 is a map of the plasmid pCR2.1poxBint.

### DETAILED DESCRIPTION OF THE INVENTION

The strains employed preferably already produce L-amino acids before amplification of the zwf gene. The term “amplification” in this connection describes the increase in the intracellular activity of one or more enzymes or proteins in a microorganism which are coded by the corresponding DNA, for example by increasing the number of copies of the gene or genes, using a potent promoter or using a gene which codes for a corresponding enzyme or protein having a high activity, and optionally combining these measures.

By amplification measures, in particular over-expression, the activity or concentration of the corresponding enzyme or protein is in general increased by at least 10%, 25%, 50%, 75%, 100%, 150%, 200%, 300%, 400% or 500%, up to a maximum of 1000% or 2000%, based on that of the wild-type enzyme or protein or the activity or concentration of the enzyme or protein in the starting microorganism.

The microorganisms which the present invention provides can prepare L-amino acids from glucose, sucrose, lactose, fructose, maltose, molasses, starch, cellulose or from glycerol and ethanol. They are representatives of coryneform bacteria, in particular of the genus Corynebacterium. Of the genus Corynebacterium, there may be mentioned in particular the species Corynebacterium glutamicum, which is known among specialists for its ability to produce L-amino acids.

Suitable strains of the genus Corynebacterium, in particular of the species Corynebacterium glutamicum, are, for example, the known wild-type strains
Corynebacterium glutamicum ATCC13032
Corynebacterium acetoglutamicum ATCC 15806
Corynebacterium acetoacidophilum ATCC 13870
Corynebacterium thermoaminogenes FERM BP-1539
Brevibacterium flavum ATCC14067
Brevibacterium lactofermentum ATCC 13869
Brevibacterium divaricatum ATCC14020
   and L-amino acid-producing mutants prepared therefrom, such as, for example, the L-threonine-producing strains
Corynebacterium glutamicum ATCC21649
Brevibacterium flavum BB69
Brevibacterium flavum DSM5399
Brevibacterium lactofermentum FERM-BP 269
Brevibacterium lactofermentum TBB-10
   and such as, for example, the L-isoleucine-producing strains
Corynebacterium glutamicum ATCC 14309
Corynebacterium glutamicum ATCC 14310
Corynebacterium glutamicum ATCC 14311
Corynebacterium glutamicum ATCC 15168
Corynebacterium ammoniagenes ATCC 6871
   and such as, for example, the L-tryptophan-producing strains
Corynebacterium glutamicum ATCC21850 and
Corynebacterium glutamicum KY9218(pKW9901)
   and such as, for example, the L-lysine-producing strains
Corynebacterium glutamicum FERM-P 1709
Brevibacterium flavum FERM-P 1708
Brevibacterium lactofermentum FERM-P 1712
Corynebacterium glutamicum FERM-P 6463
Corynebacterium glutamicum FERM-P 6464
Corynebacterium glutamicum ATCC 13032
Corynebacterium glutamicum DM58-1
Corynebacterium glutamicum DSM12866.

It has been found that coryneform bacteria produce L-amino acids, in particular L-lysine, L-threonine and L-tryptophan, in an improved manner after over-expression of the zwf gene which codes for the Zwf protein or Zwf polypeptide, respectively.

JP-A-09224661 discloses the nucleotide sequence of the zwf gene of Brevibacterium flavum MJ-223 (FERM BP-1497) and refers to the protein encoded by the zwf-gene as glucose 6-phosphate dehydrogenase. The sequence information disclosed in JP-A-09224661 is shown in SEQ ID NO:7 and 8. JP-A-09224661 describes the N-terminal amino acid sequence of the Zwf polypeptide as Met Val Ile Phe Gly Val Thr Gly Asp Leu Ala Arg Lys Lys Leu (SEQ ID NO:8).

However, it has not been possible to confirm this. Instead, the following N-terminal amino acid sequence has been found: Met Ser Thr Asn Thr Thr Pro Ser Ser Trp Thr Asn Pro Leu Arg Asp (SEQ ID NO:10). The nucleotide sequence of the corresponding zwf gene is shown in SEQ ID NO:9. The methionine residue in the N-position can be split off in the context of post-translational modification, and Ser Thr Asn Thr Thr Pro Ser Ser Trp Thr Asn Pro Leu Arg Asp is then obtained as the N-terminal amino acid sequence.

Accordingly, embodiments of this invention provide the nucleotide sequence of a novel zwf gene from a coryneform bacterium shown in SEQ ID NO:9 nucleotides 538 to 2079. Genes encoding Zwf proteins from Gram-negative bacteria e.g. Escherichia coli or other Gram-positive bacteria e.g. Streptomyces or Bacillus may optionally be used. Alleles of the zwf gene which result from the degeneracy of the genetic code or due to sense mutations of neutral function can furthermore be used. The use of endogenous genes in particular endogenous genes from coryneform bacteria is preferred. “Endogenous genes” or “endogenous nucleotide sequences” refer to genes or nucleotide sequences which are available in the population of a species.

To achieve an amplification (e.g., over-expression), the number of copies of the corresponding genes is increased, or the promoter and regulation region or the ribosome binding site upstream of the structural gene is mutated. Expression cassettes which are incorporated upstream of the structural gene act in the same way. By inducible promoters, it is additionally possible to increase the expression in the course of fermentative L-amino acid formation. The expression is likewise improved by measures to prolong the life of the m-RNA. Furthermore, the enzyme activity is also increased by preventing the degradation of the enzyme protein. The genes or gene constructs are either present here in plasmids with a varying number of copies, or are integrated and amplified in the chromosome. Alternatively, an over-expression of the genes in question can furthermore be achieved by changing the composition of the media and the culture procedure.

Instructions in this context can be found by the expert, inter alia, in Martin et al. (Bio/Technology 5, 137-146 (1987)), in Guerrero et al. (Gene 138, 35-41 (1994)), Tsuchiya and Morinaga (Bio/Technology 6, 428-430 (1988)), in Eikmanns et al. (Gene 102, 93-98 (1991)), in European Patent Specification EPS 0 472 869, in U.S. Pat. No. 4,601,893, in Schwarzer and Pühler (Bio/Technology 9, 84-87 (1991), in Reinscheid et al. (Applied and Environmental Microbiology 60, 126-132 (1994)), in LaBarre et al. (Journal of Bacteriology 175, 1001-1007 (1993)), in Patent Application WO 96/15246, in Malumbres et al. (Gene 134, 15-24 (1993)), in Japanese Laid-Open Specification JP-A-10-229891, in Jensen and Hammer (Biotechnology and Bioengineering 58, 191-195 (1998)) and in known textbooks of genetics and molecular biology.

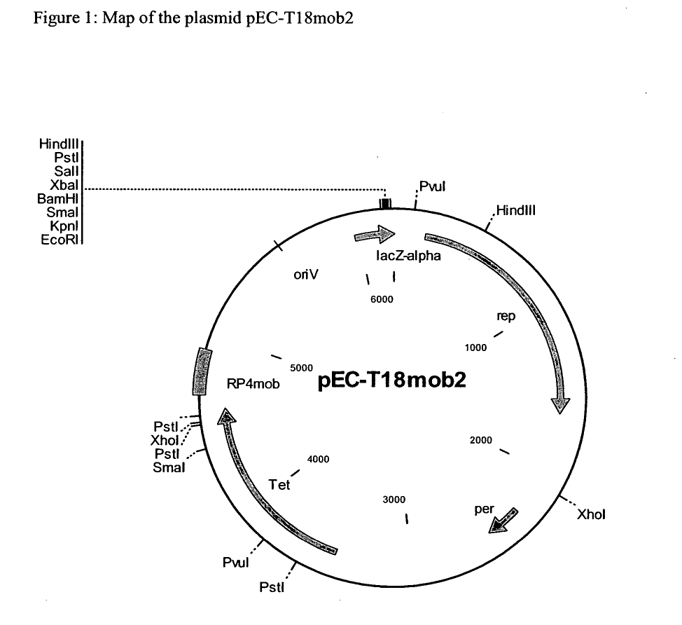

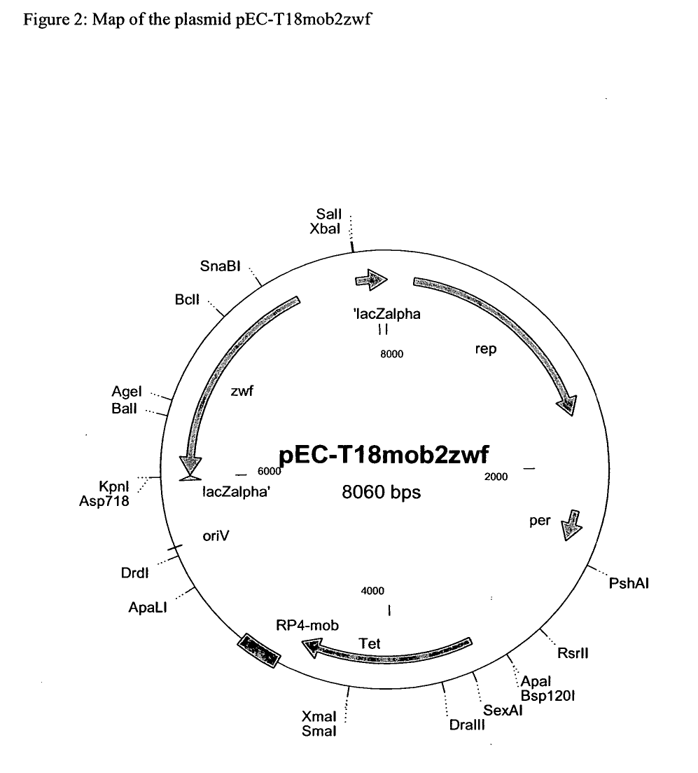

By way of example, the Zwf protein was over-expressed with the aid of a plasmid. The E. coli—C. glutamicum shuttle vector pEC-T18mob2 shown in FIG. 1 was used for this. After incorporation of the zwf gene into the KpnI/SalI cleavage site of pEC-T18mob2, the plasmid pEC-T18mob2zwf shown in FIG. 2 was formed. Other plasmid vectors which are capable of replication in C. glutamicum, such as e.g. pEKEx1 (Eikmanns et al., Gene 102:93-98 (1991)) or pZ8-1 (EP-B-0 375 889), can be used in the same way.

In addition, it may be advantageous for the production of L-amino acids to amplify one or more enzymes of the particular biosynthesis pathway, of glycolysis, of anaplerosis, of the pentose phosphate pathway or of amino acid export, in addition to amplification of the zwf gene.

Thus, for example, in particular for the preparation of L-threonine, one or more genes chosen from the group consisting of:
the hom gene which codes for homoserine dehydrogenase (Peoples et al., Molecular Microbiology 2, 63-72 (1988)) or the hom^{dr} allele which codes for a “feed back resistant” homoserine dehydrogenase (Archer et al., Gene 107, 53-59 (1991),
the gap gene which codes for glyceraldehyde 3-phosphate dehydrogenase (Eikmanns et al., Journal of Bacteriology 174: 6076-6086 (1992)),
the pyc gene which codes for pyruvate carboxylase (Peters-Wendisch et al., Microbiology 144: 915-927 (1998)),
the mqo gene which codes for malate:quinone oxidoreductase (Molenaar et al., European Journal of Biochemistry 254, 395-403 (1998)),
the tkt gene which codes for transketolase (accession number AB023377 of the European Molecular Biologies Laboratories databank (EMBL, Heidelberg, Germany)),
the gnd gene which codes for 6-phosphogluconate dehydrogenase (JP-A-9-224662),
the thrE gene which codes for the threonine export protein (DE 199 41 478.5; DSM 12840),
the zwa1 gene (DE 199 59 328.0; DSM 13115),
the eno gene which codes for enolase (DE: 199 41 478.5)
   can be amplified, in particular over-expressed, at the same time.

Thus, for example, in particular for the preparation of L-lysine, one or more genes chosen from the group consisting of:
the dapA gene which codes for dihydrodipicolinate synthase (EP-B 0 197 335),
the lysC gene which codes for a feed back resistant aspartate kinase (Kalinowski et al. (1990), Molecular and General Genetics 224: 317-324),
the gap gene which codes for glyceraldehyde 3-phosphate dehydrogenase (Eikmanns (1992), Journal of Bacteriology 174:6076-6086),
the pyc gene which codes for pyruvate carboxylase (DE-A-198 31 609),
the tkt gene which codes for transketolase (accession number AB023377 of the European Molecular Biologies Laboratories databank (EMBL, Heidelberg, Germany)),
the gnd gene which codes for 6-phosphogluconate dehydrogenase (JP-A-9-224662),
the lysE gene which codes for the lysine export protein (DE-A-195 48 222),
the zwa1 gene (DE 199 59 328.0; DSM 13115),
the eno gene which codes for enolase (DE 199 47 791.4) can be amplified, in particular over-expressed, at the same time. The use of endogenous genes is preferred.

It may furthermore be advantageous for the production of L-amino acids at the same time to attenuate one of the genes chosen from the group consisting of
the pck gene which codes for phosphoenol pyruvate carboxykinase (DE 199 50 409.1; DSM 13047),
the pgi gene which codes for glucose 6-phosphate isomerase (U.S. Ser. No. 09/396,478, DSM 12969),
the poxB gene which codes for pyruvate oxidase (DE 199 51 975.7; DSM 13114),
the zwa2 gene (DE: 199 59 327.2; DSM 13113)
   in addition to the amplification of the zwf gene.

In this connection, the term “attenuation” means reducing or suppressing the intracellular activity or concentration of one or more enzymes or proteins in a microorganism, which enzymes or proteins are coded by the corresponding DNA, for example by using a weak promoter or a gene or allele which codes for a corresponding enzyme or protein which has a low activity or inactivates the corresponding enzyme or protein and optionally by combining these measures.

By attenuation measures, the activity or concentration of the corresponding enzyme or protein is in general reduced to 0 to 75%, 0 to 50%, 0 to 25%, 0 to 10% or 0 to 5% of the activity or concentration of the wild-type enzyme or protein or of the activity or concentration of the enzyme or protein in the starting microorganism.

In addition to over-expression of the Zwf protein, it may furthermore be advantageous for the production of L-amino acids to eliminate undesirable side reactions (Nakayama: “Breeding of Amino Acid Producing Micro-organisms”, in: Overproduction of Microbial Products, Krumphanzl, Sikyta, Vanek (eds.), Academic Press, London, UK, 1982).

The microorganisms prepared according to the invention can be cultured continuously or discontinuously in the batch process (batch culture) or in the fed batch (feed process) or repeated fed batch process (repetitive feed process) for the purpose of L-amino acid production. A summary of known culture methods is described in the textbook by Chmiel (Bioprozesstechnik 1. Einführung in die Bioverfahrenstechnik [Bioprocess Technology 1. Introduction to Bioprocess Technology (Gustav Fischer Verlag, Stuttgart, 1991)) or in the textbook by Storhas (Bioreaktoren und periphere Einrichtungen [Bioreactors and Peripheral Equipment] (Vieweg Verlag, Braunschweig[Wiesbaden, 1994)).

The culture medium to be used should meet the requirements of the particular microorganisms in a suitable manner. Descriptions of culture media for various microorganisms are contained in the handbook “Manual of Methods for General Bacteriology” of the American Society for Bacteriology (Washington D.C., USA, 1981). Sugars and carbohydrates, such as e.g. glucose, sucrose, lactose, fructose, maltose, molasses, starch and cellulose, oils and fats, such as e.g. soya oil, sunflower oil, groundnut oil and coconut fat, fatty acids, such as e.g. palmitic acid, stearic acid and linoleic acid, alcohols, such as, e.g., glycerol and ethanol, and organic acids, such as e.g. acetic acid, can be used as the source of carbon. These substance can be used individually or as a mixture. Organic nitrogen-containing compounds, such as peptones, yeast extract, meat extract, malt extract, corn steep liquor, soya bean flour and urea, or inorganic compounds, such as ammonium sulfate, ammonium chloride, ammonium phosphate, ammonium carbonate and ammonium nitrate, can be used as the source of nitrogen. The sources of nitrogen can be used individually or as a mixture. Potassium dihydrogen phosphate or dipotassium hydrogen phosphate or the corresponding sodium-containing salts can be used as the source of phosphorus. The culture medium should furthermore comprise salts of metals, such as e.g. magnesium sulfate or iron sulfate, which are necessary for growth. Finally, essential growth substances, such as amino acids and vitamins, can be employed in addition to the above-mentioned substances. Suitable precursors can moreover be added to the culture medium. The starting substances mentioned can be added to the culture in the form of a single batch, or can be fed in during the culture in a suitable manner.

Basic compounds, such as sodium hydroxide, potassium hydroxide, ammonia, or acid compounds, such as phosphoric acid or sulfuric acid, can be employed in a suitable manner to control the pH. Antifoams, such as e.g. fatty acid polyglycol esters, can be employed to control the development of foam. Suitable substances having a selective action, e.g. antibiotics, can be added to the medium to maintain the stability of plasmids. To maintain aerobic conditions, oxygen or oxygen-containing gas mixtures, such as e.g. air, are introduced into the culture. The temperature of the culture is usually 20° C. to 45° C., and preferably 25° C. to 40° C. Culturing is continued until a maximum of L-amino acid has formed. This target is usually reached within 10 hours to 160 hours.

The analysis of L-amino acids can be carried out by anion exchange chromatography with subsequent ninhydrin derivation, as described by Spackman et al. (Analytical Chemistry, 30, (1958), 1190), or it can take place by reversed phase HPLC as described by Lindroth et al. (Analytical Chemistry (1979) 51:. 1167-1174).

The following microorganism has been deposited at the Deutsche Sammlung für Mikroorganismen und Zellkulturen (DSMZ=German Collection of Microorganisms and Cell Cultures, Braunschweig, Germany) in accordance with the Budapest Treaty: Escherichia coli K-12 DH5α/pEC-T18mob2 as DSM 13244.

Referring now more particularly to the Figures, in FIGS. 1 and 2, the abbreviations used have the following meanings:

| | |
|---|---|
| Tet: | Resistance gene for tetracycline |
| oriV: | Plasmid-coded replication origin of E. coli |
| RP4mob: | mob region for mobilizing the plasmid |
| rep: | Plasmid-coded replication origin from C. glutamicum |
| | plasmid pGA1 |
| per: | Gene for controlling the number of copies from pGA1 |
| lacZ-alpha: | lacZα gene fragment (N-terminus) of the |
| | β-galactosidase gene |
| lacZalpha': | 5′-Terminus of the lacZα gene fragment |
| 'lacZalpha: | 3′-Terminus of the lacZα gene fragment |

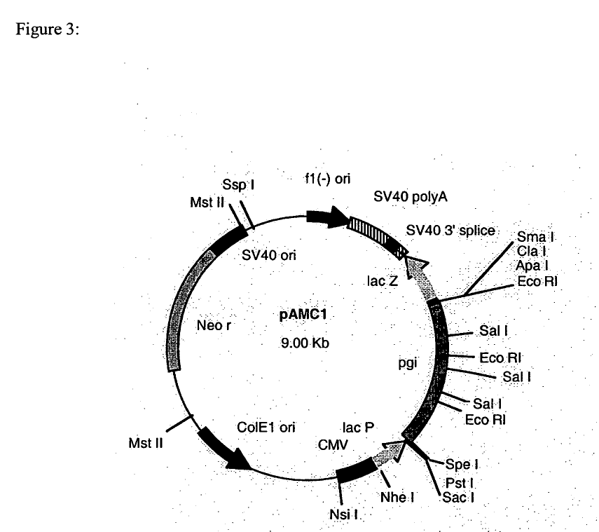

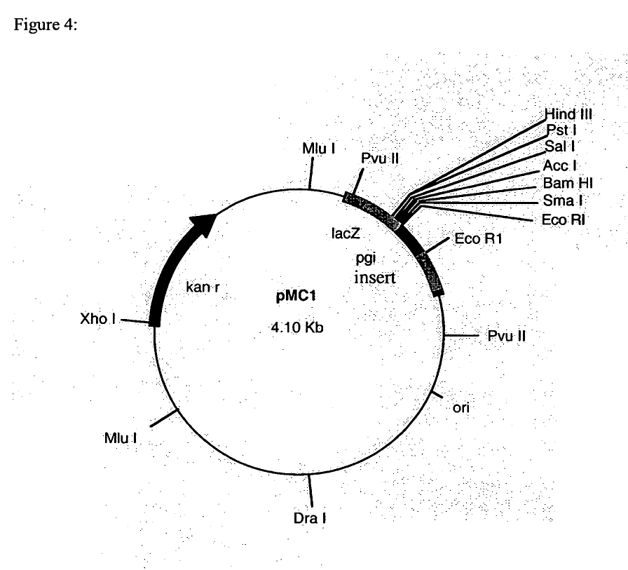

In FIGS. 3 and 4, the abbreviations used have the following meanings:

| | |
|---|---|
| Neo r: | Neomycin/kanamycin resistance |
| ColE1 ori: | Replication origin of the plasmid ColE1 |
| CMV: | Cytomegalovirus promoter |
| lacP: | Lactose promoter |
| pgi: | Phosphoglucose isomerase gene |
| lacZ: | Part of the β-galactosidase gene |
| SV40 3′ splice | 3′ splice site of Simian virus 40 |
| SV40 polyA: | Polyadenylation site of Simian virus 40 |
| f1(-)ori: | Replication origin of the filamentous phage f1 |
| SV40 ori: | Replication origin of Simian virus 40 |
| kan r: | Kanamycin resistance |
| pgi insert: | Internal fragment of the pgi gene |
| ori: | Replication origin of the plasmid pBGS8 |

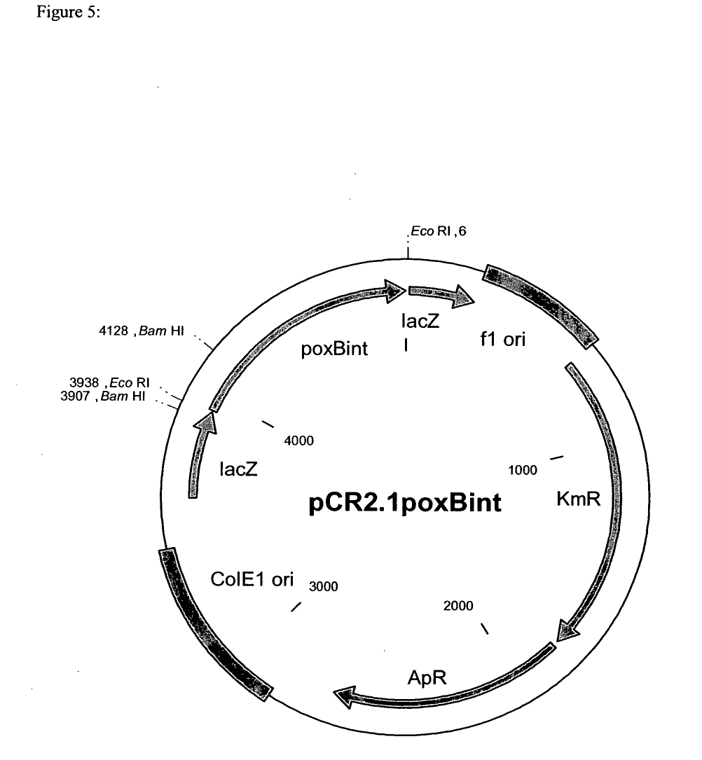

In FIG. 5, the abbreviations used have the following meanings:

| | |
|---|---|
| ColE1 ori: | Replication origin of the plasmid ColE1 |
| lacZ: | Cloning relict of the lacZα gene fragment |
| f1 ori: | Replication origin of phage f1 |
| KmR: | Kanamycin resistance |
| ApR: | Ampicillin resistance |
| poxBint: | Internal fragment of the poxB gene. |

The meaning of the abbreviations for the various restriction enzymes (e.g. BamHI, EcoRI etc.) are known from the prior art and are summarized, for example, by Kessler and Höltke (Gene 47, 1-153 (1986)) or Roberts et al. (Nucleic Acids Research 27, 312-313 (1999)).

The following examples will further illustrate this invention. The molecular biology techniques, e.g. plasmid DNA isolation, restriction enzyme treatment, ligations, standard transformations of Escherichia coli etc. used are, (unless stated otherwise), described by Sambrook et al., (Molecular Cloning. A Laboratory Manual (1989) Cold Spring Harbor Laboratories, USA).

### EXAMPLE 1

### Expression of the Zwf Protein

1.1 Preparation of the plasmid pEC-T 18mob2

The E. coli—C. glutamicum shuttle vector pEC-T18mob2 was constructed according to the prior art. The vector contains the replication region rep of the plasmid pGA1 including the replication effector per (U.S. Pat. No. 5,175,108; Nesvera et al., Journal of Bacteriology 179, 1525-1532 (1997)), the tetracycline resistance-imparting tetA(Z) gene of the plasmid pAGI (U.S. Pat. No. 5,158,891; gene library entry at the National Center for Biotechnology Information (NCBI, Bethesda, Md., USA) with accession number AF121000), the replication region oriV of the plasmid pMB1(Sutcliffe, Cold Spring Harbor Symposium on Quantitative Biology 43, 77-90 (1979)), the lacZα gene fragment including the lac promoter and a multiple cloning site (mcs) (Norrander et al. Gene 26, 101-106 (1983)) and the mob region of the plasmid RP4 (Simon et al., (1983) Bio/Technology 1:784-791). The vector constructed was transformed in the E. coli strain DH5α (Brown (ed.) Molecular Biology Labfax, BIOS Scientific Publishers, Oxford, UK, 1991). Selection for plasmid-carrying cells was made by plating out the transformation batch on LB agar (Sambrook et al., Molecular cloning: a laboratory manual. 2^{nd} Ed. Cold Spring Harbor Laboratory Press, Cold Spring Harbor, N.Y.), which had been supplemented with 5 mg/l tetracycline. Plasmid DNA was isolated from a transformant with the aid of the QIAprep Spin Miniprep Kit from Qiagen and checked by restriction with the restriction enzyme EcoRI and HindIII and subsequent agarose gel electrophoresis (0.8%).

The plasmid was called pEC-T18mob2 and is shown in FIG. 1. It is deposited in the form of the strain Escherichia coli K-12 strain DH5αpEC-T18mob2 at the Deutsche Sammlung für Mikroorganismen und Zellkulturen (DSMZ=German Collection of Microorganisms and Cell Cultures, Braunschweig, Germany) as DSM 13244.

1.2 Preparation of the Plasmid pEC-T18mob2zwf

The gene from Corynebacterium glutamicum ATCC13032 was first amplified by a polymerase chain reaction (PCR) by means of the following oligonucleotide primer:

| | |
|---|---|
| zwf-forward: | |
| 5′-TCG ACG CGG TTC TGG AGC AG-3′ | (SEQ ID NO:11) |
| zwf-reverse: | |
| 5′-CTA AAT TAT GGC CTG CGC CAG-3′. | (SEQ ID NO:12) |

The PCR reaction was carried out in 30 cycles in the presence of 200 μM deoxynucleotide triphosphates (dATP, dCTP, dGTP, dTTP), in each case 1 μM of the corresponding oligonucleotide, 100 ng chromosomal DNA from Corynebacterium glutamicum ATCC13032, 1/10 volume 10-fold reaction buffer and 2.6 units of a heat-stable Taq-/Pwo-DNA polymerase mixture (Expand High Fidelity PCR System from Roche Diagnostics, Mannheim, Germany) in a Thermocycler (PTC-100, MJ Research, Inc., Watertown, USA) under the following conditions: 94° C. for 30 seconds, 64° C. for 1 minute and 68° C. for 3 minutes.

The amplified fragment about 1.8 kb in size was subsequently ligated with the aid of the SureClone Ligation Kit (Amersham Pharmacia Biotech, Uppsala, Sweden) into the SmaI cleavage site of the vector pUC18 in accordance with the manufacturer's instructions. The E. coli strain DH5αmcr (Grant et al., Proceedings of the National Academy of Sciences of the United States of America USA (1990) 87: 4645-4649) was transformed with the entire ligation batch. Transformants were identified with the aid of their carbenicillin resistance on LB-agar plates containing 50 μg/mL carbenicillin. The plasmids were prepared from 7 of the transformants and checked for the presence of the 1.8 kb PCR fragment as an insert by restriction analysis. The recombinant plasmid formed in this way is called pUC18zwf in the following.

For construction of pEC-T18mob2zwf, pUC18zwf was digested with KpnI and SalI, and the product was isolated with the aid of the NucleoSpin Extraction Kit from Macherey-Nagel (Düren, Germany) in accordance with the manufacturer's instructions and then ligated with the vector pEC-T18mob2, which had also been cleaved with KpnI and SalI and dephosphorylated. The E. coli strain DH5αmcr (Grant et al., Proceedings of the National Academy of Sciences of the United States of America USA (1990) 87: 4645-4649) was transformed with the entire ligation batch. Transformants were identified with the aid of their tetracycline resistance on LB-agar plates containing 5 μg/mL tetracycline. The plasmids were prepared from 12 of the transformants and checked for the presence of the 1.8 kb PCR fragment as an insert by restriction analysis. One of the recombinant plasmids isolated in this manner was called PEC-T18mob2zwf (FIG. 2).

### EXAMPLE 2

### Preparation of Amino Acid Producers with an Amplified zwf Gene

The L-lysine-producing strain Corynebacterium glutamicum DSM5715 is described in EP-B-0435132 and the L-threonine-producing strain Brevibacterium flavum DSM5399 is described in EP-B-0385940. Both strains are deposited at the Deutsche Sammlung für Mikroorganismen und Zellkulturen [German Collection of Microorganisms and Cell Cultures] in Braunschweig (Germany) in accordance with the Budapest Treaty.

2.1 Preparation of the Strains DSM5715/pEC-T18mob2zwf and DSM5399/pEC-T18mob2zwf

The strains DSM5715 and DSM5399 were transformed with the plasmid pEC-T18mob2zwf using the electroporation method described by Liebl et al., (FEMS Microbiology Letters, 53:299-303 (1989)) Selection of the transformants took place on LBHIS agar comprising 18.5 g/l brain-heart infusion broth, 0.5 M sorbitol, 5 g/l Bacto-tryptone, 2.5 g/l Bacto-yeast extract, 5 g/l NaCl and 18 g/l Bacto-agar, which had been supplemented with 5 mg/l tetracycline. Incubation was carried out for 2 days at 33° C.

Plasmid DNA was isolated in each case from a transformant by conventional methods (Peters-Wendisch et al., 1998, Microbiology 144, 915-927), cleaved with the restriction endonucleases XbaI and KpnI, and the plasmid was checked by subsequent agarose gel electrophoresis. The strains obtained in this way were called DSM5715/pEC-T18mob2zwf and DSM5399/pEC-T18mob2zwf.

2.2 Preparation of L-threonine

The C. glutamicum strain DSM5399/pEC-T18mob2zwf obtained in Example 2.1 was cultured in a nutrient medium suitable for the production of threonine and the threonine content in the culture supernatant was determined. For this, the strain was first incubated on an agar plate with the corresponding antibiotic (brain-heart agar with tetracycline (5 mg/l)) for 24 hours at 33° C. Starting from this agar plate culture, a preculture was seeded (10 ml medium in a 100 ml conical flask). The complete medium Cg III was used as the medium for the preculture.

### Medium Cg III

| | |
|---|---|
| NaCl | 2.5 g/l |
| Bacto-Peptone | 10 g/l |
| Bacto-Yeast extract | 10 g/l |
| Glucose (autoclaved separately) | 2% (w/v) |

The pH was brought to pH 7.4

Tetracycline (5 mg/l) was added to this. The preculture was incubated for 16 hours at 33° C. at 240 rpm on a shaking machine. A main culture was seeded from this preculture such that the initial OD (660 nm) of the main culture was 0.1. Medium MM was used for the main culture.

### Medium MM

| | |
|---|---|
| CSL (corn steep liquor) | 5 g/l |
| MOPS (morpholinopropanesulfonic acid) | 20 g/l |
| Glucose (autoclaved separately) | 50 g/l |
| (NH₄)₂SO₄ | 25 g/l |
| KH₂PO₄ | 0.1 g/l |
| MgSO₄ * 7 H₂O | 1.0 g/l |
| CaCl₂ * 2 H₂O | 10 mg/l |
| FeSO₄ * 7 H₂O | 10 mg/l |
| MnSO₄ * H₂O | 5.0 mg/l |
| Biotin (sterile-filtered) | 0.3 mg/l |
| Thiamine * HCl (sterile-filtered) | 0.2 mg/l |
| L-Leucine (sterile-filtered) | 0.1 g/l |
| CaCO₃ | 25 g/l |

The CSL, MOPS and the salt solution were brought to pH 7 with aqueous ammonia and autoclaved. The sterile substrate and vitamin solutions were then added, as well as the CaCO₃ autoclaved in the dry state. Culturing is carried out in a 10 ml volume in a 100 ml conical flask with baffles. Tetracycline (5 mg/l) was added. Culturing was carried out at 33° C. and 80% atmospheric humidity.

After 72 hours, the OD was determined at a measurement wavelength of 660 nm with a Biomek 1000 (Beckmann Instruments GmbH, Munich). The amount of threonine formed was determined with an amino acid analyzer from Eppendorf-BioTronik (Hamburg, Germany) by ion exchange chromatography and post-column derivation with ninhydrin detection. The result of the experiment is shown in Table 1.

TABLE 1

| | OD | L-Threonin |
|---|---|---|
| Strain | (660 nm) | g/l |
| DSM5399 | 12.3 | 0.74 |
| DSM5399/pEC-T18mob2zwf | 10.2 | 1.0 |

2.3 Preparation of L-lysine

The C. glutamicum strain DSM5715/pEC-T18mob2zwf obtained in Example 2.1 was cultured in a nutrient medium suitable for the production of lysine and the lysine content in the culture supernatant was determined. For this, the strain was first incubated on an agar plate with the corresponding antibiotic (brain-heart agar with tetracycline (5 mg/l)) for 24 hours at 33° C. Starting from this agar plate culture, a preculture was seeded (10 ml medium in a 100 ml conical flask). The complete medium Cg III was used as the medium for the preculture.

### Medium Cg III

| | |
|---|---|
| NaCl | 2.5 g/l |
| Bacto-Peptone | 10 g/l |
| Bacto-Yeast extract | 10 g/l |
| Glucose (autoclaved separately) | 2% (w/v) |

The pH was brought to pH 7.4

Tetracycline (5 mg/l) was added to this. The preculture was incubated for 16 hours at 33° C. at 240 rpm on a shaking machine. A main culture was seeded from this preculture such that the initial OD (660 nm) of the main culture was 0.1. Medium MM was used for the main culture.

### Medium MM

| | |
|---|---|
| CSL (corn steep liquor) | 5 g/l |
| MOPS (morpholinopropanesulfonic acid) | 20 g/l |
| Glucose (autoclaved separately) | 58 g/l |
| (NH₄)₂SO₄ | 25 g/l |
| KH₂PO₄ | 0.1 g/l |
| MgSO₄ * 7 H₂O | 1.0 g/l |
| CaCl₂ * 2 H₂O | 10 mg/l |
| FeSO₄ * 7 H₂O | 10 mg/l |
| MnSO₄ * H₂O | 5.0 mg/l |
| Biotin (sterile-filtered) | 0.3 mg/l |
| Thiamine * HCl (sterile-filtered) | 0.2 mg/l |
| L-Leucine (sterile-filtered) | 0.1 g/l |
| CaCO₃ | 25 g/l |

The CSL, MOPS and the salt solution were brought to pH 7 with aqueous ammonia and autoclaved. The sterile substrate and vitamin solutions were then added, as well as the CaCO₃ autoclaved in the dry state. Culturing is carried out in a 10 ml volume in a 100 ml conical flask with baffles. Tetracycline (5 mg/l) was added. Culturing was carried out at 33° C. and 80% atmospheric humidity. After 72 hours, the OD was determined at a measurement wavelength of 660 nm with a Biomek 1000 (Beckmann Instruments GmbH, Munich). The amount of lysine formed was determined with an amino acid analyzer from Eppendorf-BioTronik (Hamburg, Germany) by ion exchange chromatography and post-column derivation with ninhydrin detection. The result of the experiment is shown in Table 2.

TABLE 2

| | OD | L-Lysine HCl |
|---|---|---|
| Strain | (660 nm) | g/l |
| DSM5715 | 10.8 | 16.0 |
| DSM5715/pEC-T18mob2zwf | 7.2 | 17.1 |

### EXAMPLE 3

### Construction of a Gene Library of Corynebacterium glutamicum Strain AS019

A DNA library of Corynebacterium glutamicum strain ASO19 (Yoshihama et al., Journal of Bacteriology 162, 591-597 (1985)) was constructed using λ Zap Express™ system, (Short et al., (1988) Nucleic Acids Research, 16: 7583-7600), as described by O'Donohue (O'Donohue, M. (1997). The Cloning and Molecular Analysis of Four Common Aromatic Amino Acid Biosynthetic Genes from Corynebacterium glutamicum. Ph.D. Thesis, National University of Ireland, Galway). λ Zap Express™ kit was purchased from Stratagene (Stratagene, 11011 North Torrey Pines Rd., La Jolla, Calif. 92037) and used according to the manufacturers instructions. AS019-DNA was digested with restriction enzyme Sau3A and ligated to BamHI treated and dephosphorylated λ Zap Express™ arms.

### EXAMPLE 4

### Cloning and Sequencing of the pgi Gene

1. Cloning

Escherichia coli strain DF1311, carrying mutations in the pgi and pgl genes as described by Kupor and Fraenkel, (Journal of Bacteriology 100: 1296-1301 (1969)), was transformed with approx. 500 ng of the AS019 λ Zap Express™ plasmid library described in Example 3. Selection for transformants was made on M9 minimal media, (Sambrook et al., (1989). Molecular Cloning. A Laboratory Manual Cold Spring Harbor Laboratories, USA), containing kanamycin at a concentration of 50 mg/l and incubation at 37° C. for 48 hours. Plasmid DNA was isolated from one transformant according to Birnboim and Doly (Nucleic Acids Research 7: 1513-1523 (1979)) and designated pAMC1 (FIG. 3).

2. Sequencing

For sequence analysis of the cloned insert of pAMC1 the method of Sanger et al. (Proceedings of the National Academy of Sciences USA 74,5463-5467 (1977)) was applied using primers differentially labeled with a colored fluorescent tag. It was carried out using the ABI prism 310 genetic analyzer from Perkin Elmer Applied Biosystems, (Perkin Elmer Corporation, Norwalk, Conn., U.S.A), and the ABI prism Big Dye™ Terminator Cycle Sequencing Ready Reaction kit also from Perkin Elmer.

Initial sequence analysis was carried out using the universal forward and M13 reverse primers obtained from Pharmacia Biotech (St. Albans, Herts, AL13AW, UK):

| | |
|---|---|
| Universal forward primer: | |
| GTA ATA CGA CTC ACT ATA GGG C | (SEQ ID NO:13) |
| M13 reverse primer: | |
| GGA AAC AGC TAT GAG CAT G. | (SEQ ID NO:14) |

Internal primers were subsequently designed from the sequence obtained which allowed the entire pgi gene to be deduced. The sequence of the internal primers is as follows:

| | |
|---|---|
| Internal primer 1: | |
| GGA AAC AGG GGA GCC GTC | (SEQ ID NO:15) |
| Internal primer 2: | |
| TGC TGA GAT ACC AGC GGT. | (SEQ ID NO:16) |

The sequence obtained was then analyzed using the DNA Strider program, (Marck, (1988). Nucleic Acids Research 16: 1829-1836), version 1.0 on an Apple Macintosh computer. This program allowed for analyses such as restriction site usage, open reading frame analysis and codon usage determination. Searches between DNA sequence obtained and those in EMBL and Genbank databases were achieved using the BLAST program, (Altschul et al., (1997). Nucleic Acids Research, 25: 3389-3402). DNA and protein sequences were aligned using the Clustal V and Clustal W programs (Higgins and Sharp, 1988 Gene 73: 237-244).

The sequence thus obtained is shown in SEQ ID NO:1. The analysis of the nucleotide sequence obtained revealed an open reading frame of 1650 base pairs which was designated as pgi gene. It codes for a protein of 550 amino acids shown in SEQ ID NO:2.

### EXAMPLE 5

### Preparation of an Integration Vector for Integration Mutagenesis of the pgi Gene

An internal segment of the pgi gene was amplified by polymerase chain reaction (PCR) using genomic DNA isolated from Corynebacterium glutamicum AS019, (Heery and Dunican, (1993) Applied and Environmental Microbiology 59: 791-799), as template. The pgi primers used were:

| | |
|---|---|
| fwd. Primer: | |
| ATG GAR WCC AAY GGH AA | (SEQ ID NO:17) |
| rev. Primer: | |
| YTC CAC GCC CCA YTG RTC | (SEQ ID NO:18) |

with R=A+G; Y═C+T; W=A+T; H=A+T+C.
PCR Parameters were as follows: 35 cycles
94° C. for 1 min.
47° C. for 1 min.
72° C. for 30 sec.
1.5 mM MgCl₂
approx. 150-200 ng DNA template.

The PCR product obtained was cloned into the commercially available pGEM-T vector received from Promega Corp., (Promega UK, Southampton.) using strain E. coli JM109, (Yanisch-Perron et al., 1985. Gene, 33: 103-119), as a host. The sequence of the PCR product is shown as SEQ ID NO:3. The cloned insert was then excised as an EcoRI fragment and ligated to plasmid pBGS8 (Spratt et al., Gene 41: 337-342 (1986)) pretreated with EcoRI. The restriction enzymes used were obtained from Boehringer Mannheim UK Ltd., (Bell Lane, Lewes East Sussex BN7 1LG, UK.) and used according to manufacturers instructions. E. coli JM109 was then transformed with this ligation mixture and electrotransformants were selected on Luria agar supplemented with IPTG (isopropyl-β-D-thiogalactopyranoside), XGAL (5-bromo-4-chloro-3-indolyl-D-galactopyranoside) and kanamycin at a concentration of 1 mM, 0.02% and 50 mg/l respectively. Agar plates were incubated for twelve hours at 37° C. Plasmid DNA was isolated from one transformant, characterized by restriction enzyme analysis using EcoRI, BamHI and SalI designated pMC1 (FIG. 4).

Plasmid pMC1 was deposited in the form of Escherichia coli strain DH5α/pMC1 at the Deutsche Sammlung für Mikroorganismen und Zellkulturen (DSMZ, Braunschweig, Germany) as DSM 12969 according to the Budapest treaty.

### EXAMPLE 6

### Integration Mutagenesis of the pgi Gene in the Lysine Producer DSM 5715

The vector pMC1 mentioned in Example 5 was electroporated by the electroporation method of Tauch et al. (FEMS Microbiological Letters, 123:343-347 (1994)) in Corynebacterium glutamicum DSM 5715. Strain DSM 5715 is an AEC-resistant lysine producer. The vector pMC1 cannot replicate independently in DSM5715 and is retained in the cell only if it has integrated into the chromosome of DSM 5715. Selection of clones with pMC1 integrated into the chromosome was carried out by plating out the electroporation batch on LB agar (Sambrook et al., Molecular cloning: a laboratory manual. 2^{nd} Ed. Cold Spring Harbor Laboratory Press, Cold Spring Harbor, N.Y.), which had been supplemented with 15 mg/] kanamycin. For detection of the integration, the internal pgi fragment (Example 5) was labeled with the Dig hybridization kit from Boehringer Mannheim by the method of “The DIG System Users Guide for Filter Hybridization” of Boehringer Mannheim GmbH (Mannheim, Germany, 1993). Chromosomal DNA of a transformant was isolated by the method of Eikmanns et al. (Microbiology 140: 1817-1828 (1994)) and in each case cleaved with the restriction enzymes SalI, SacI and HindIII. The fragments formed were separated by agarose gel electrophoresis and hybridized at 68° C. with the Dig hybridization kit from Boehringer. It was found in this way that the plasmid pMC1 was inserted within the chromosomal pgi gene of strain DSM5715. The strain was called DSM5715::pMC1.

### EXAMPLE 7

### Effect of Over-Expression of the zwf Gene with Simultaneous Elimination of the pgi Gene on the Preparation of Lysine

7.1 Preparation of the Strain DSM5715::pMC1/pEC-T 18mob2zwf

The vector pEC-T18mob2zwf mentioned in Example 1.2 was electroporated by the electroporation method of Tauch et al. (1994, FEMS Microbiological Letters, 123:343-347) in Corynebacterium glutamicum DSM 5715::pMC1. Selection for plasmid-carrying cells was made by plating out the electroporation batch on LB agar (Sambrook et al., Molecular cloning: a laboratory manual. 2^{nd} Ed. Cold Spring Harbor Laboratory Press, Cold Spring Harbor, N.Y., 1989), which had been supplemented with 15 mg/l kanamycin and with 5 mg/] tetracycline. Plasmid DNA was isolated from a transformant by conventional methods (Peters-Wendisch et al., 1998, Microbiology 144, 915-927) and checked by treatment with the restriction enzymes KpnI and SalI with subsequent agarose gel electrophoresis. The strain was called DSM5715::pMC1/pEC-T18mob2zwf.

7.2 Preparation of Lysine

The C. glutamicum strain DSM5715::pMC1/pEC-T18mob2zwf obtained in Example 7.1 was cultured in a nutrient medium suitable for the production of lysine and the lysine content in the culture supernatant was determined. For this, the strain was first incubated on an agar plate with the corresponding antibiotic (brain-heart agar with tetracycline (5 mg/l) and kanamycin (25 mg/l)) for 24 hours at 33° C. The cultures of the comparison strains were supplemented according to their resistance to antibiotics. Starting from this agar plate culture, a preculture was seeded (10 ml medium in a 100 ml conical flask). The complete medium Cg III was used as the medium for the preculture.

### Medium Cg III

| | |
|---|---|
| NaCl | 2.5 g/l |
| Bacto-Peptone | 10 g/l |
| Bacto-Yeast extract | 10 g/l |
| Glucose (autoclaved separately) | 2% (w/v) |

The pH was brought to pH 7.4

Tetracycline (5 mg/l) and kanamycin (5 mg/l) was added to this. The preculture was incubated for 16 hours at 33° C. at 240 rpm on a shaking machine. A main culture was seeded from this preculture such that the initial OD (660 nm) of the main culture was 0.1. Medium MM was used for the main culture.

### Medium MM

| | |
|---|---|
| CSL (corn steep liquor) | 5 g/l |
| MOPS (morpholinopropanesulfonic acid) | 20 g/l |
| Glucose (autoclaved separately) | 50 g/l |
| (NH₄)₂SO₄ | 25 g/l |
| KH₂PO₄ | 0.1 g/l |
| MgSO₄ * 7 H₂O | 1.0 g/l |
| CaCl₂ * 2 H₂O | 10 mg/l |
| FeSO₄ * 7 H₂O | 10 mg/l |
| MnSO₄ * H₂O | 5.0 mg/l |
| Biotin (sterile-filtered) | 0.3 mg/l |
| Thiamine * HCl (sterile-filtered) | 0.2 mg/l |
| L-Leucine (sterile-filtered) | 0.1 g/l |
| CaCO₃ | 25 g/l |

The CSL, MOPS and the salt solution were brought to pH 7 with aqueous ammonia and autoclaved. The sterile substrate and vitamin solutions were then added, as well as the CaCO₃ autoclaved in the dry state. Culturing is carried out in a 10 ml volume in a 100 ml conical flask with baffles. Tetracycline (5 mg/l) and kanamycin (25 mg/l) were added. Culturing was carried out at 33° C. and 80% atmospheric humidity.

After 72 hours, the OD was determined at a measurement wavelength of 660 nm with a Biomek 1000 (Beckmann Instruments GmbH, Munich). The amount of lysine formed was determined with an amino acid analyzer from Eppendorf-BioTronik (Hamburg, Germany) by ion exchange chromatography and post-column derivation with ninhydrin detection. The result of the experiment is shown in Table 3.

TABLE 3

| | OD | L-Lysine HCl |
|---|---|---|
| Strain | (660 nm) | g/l |
| DSM5715 | 7.3 | 14.3 |
| DSM5715/pEC-T18mob2zwf | 7.1 | 14.6 |
| DSM5715::pMC1/ | 10.4 | 15.2 |
| pECTmob2zwf | | |

### EXAMPLE 8

### Preparation of a Genomic Cosmid Gene Library from Corynebacterium glutamicum ATCC 13032

Chromosomal DNA from Corynebacterium glutamicum ATCC 13032 was isolated as described by Tauch et al., (1995, Plasmid 33:168-179), and partly cleaved with the restriction enzyme Sau3AI (Amersham Pharmacia, Freiburg, Germany, Product Description Sau3AI, Code no. 27-0913-O₂). The DNA fragments were dephosphorylated with shrimp alkaline phosphatase (Roche Molecular Biochemicals, Mannheim, Germany, Product Description SAP, Code no. 1758250). The DNA of the cosmid vector SuperCosl (Wahl et al.

Proceedings of the National Academy of Sciences USA 84:2160-2164), obtained from Stratagene (La Jolla, USA, Product Description SuperCosl Cosmid Vektor Kit, Code no. 251301) was cleaved with the restriction enzyme XbaI (Amersham Pharmacia, Freiburg, Germany, Product Description XbaI, Code no. 27-0948-O₂) and likewise dephosphorylated with shrimp alkaline phosphatase. The cosmid DNA was then cleaved with the restriction enzyme BamHI (Amersham Pharmacia, Freiburg, Germany, Product Description BamHI, Code no. 27-0868-04). The cosmid DNA treated in this manner was mixed with the treated ATCC13032 DNA and the batch was treated with T4 DNA ligase (Amersham Pharmacia, Freiburg, Germany, Product Description T4-DNA-Ligase, Code no.27-0870-04). The ligation mixture was then packed in phages with the aid of Gigapack II XL Packing Extracts (Stratagene, La Jolla, USA, Product Description Gigapack II XL Packing Extract, Code no. 200217). For infection of the E. coli strain NM554 (Raleigh et al. 1988, Nucleic Acid Res. 16:1563-1575) the cells were taken up in 10 mM MgSO₄ and mixed with an aliquot of the phage suspension. The infection and titering of the cosmid library were carried out as described by Sambrook et al. (1989, Molecular Cloning: A laboratory Manual, Cold Spring Harbor), the cells being plated out on LB agar (Lennox, 1955, Virology, 1:190)+100 μg/ml ampicillin. After incubation overnight at 37° C., recombinant individual clones were selected.

### EXAMPLE 9

### Isolation and Sequencing of the poxB Gene

The cosmid DNA of an individual colony (Example 7) was isolated with the Qiaprep Spin Miniprep Kit (Product No. 27106, Qiagen, Hilden, Germany) in accordance with the manufacturer's instructions and partly cleaved with the restriction enzyme Sau3AI (Amersham Pharmacia, Freiburg, Germany, Product Description Sau3AI, Product No. 27-0913-02). The DNA fragments were dephosphorylated with shrimp alkaline phosphatase (Roche Molecular Biochemicals, Mannheim, Germany, Product Description SAP, Product No. 1758250). After separation by gel electrophoresis, the cosmid fragments in the size range of 1500 to 2000 bp were isolated with the QiaExII Gel Extraction Kit (Product No. 20021, Qiagen, Hilden, Germany). The DNA of the sequencing vector pZero-1, obtained from Invitrogen (Groningen, Holland, Product Description Zero Background Cloning Kit, Product No. K2500-01), was cleaved with the restriction enzyme BamHI (Amersham Pharmacia, Freiburg, Germany, Product Description BamHI, Product No. 27-0868-04). The ligation of the cosmid fragments in the sequencing vector pZero-1 was carried out as described by Sambrook et al. (1989, Molecular Cloning: A laboratory Manual, Cold Spring Harbor), the DNA mixture being incubated overnight with T4 ligase (Pharmacia Biotech, Freiburg, Germany). This ligation mixture was then electroporated (Tauch et al. 1994, FEMS Microbiol Letters, 123:343-7) into the E. coli strain DH5αMCR (Grant, 1990, Proceedings of the National Academy of Sciences U.S.A., 87:4645-4649) and plated out on LB agar (Lennox, 1955, Virology, 1:190) with 50 μg/ml zeocin. The plasmid preparation of the recombinant clones was carried out with Biorobot 9600 (Product No. 900200, Qiagen, Hilden, Germany). The sequencing was carried out by the dideoxy chain-stopping method of Sanger et al. (1977, Proceedings of the National Academies of Sciences U.S.A., 74:5463-5467) with modifications according to Zimmermann et al. (1990, Nucleic Acids Research, 18:1067). The “RR dRhodamin Terminator Cycle Sequencing Kit” from PE Applied Biosystems (Product No. 403044, Weiterstadt, Germany) was used. The separation by gel electrophoresis and analysis of the sequencing reaction were carried out in a “Rotiphoresis NF Acrylamide/Bisacrylamide” Gel (29:1) (Product No. A124.1, Roth, Karlsruhe, Germany) with the “ABI Prism 377” sequencer from PE Applied Biosystems (Weiterstadt, Germany).

The raw sequence data obtained were then processed using the Staden program package (1986, Nucleic Acids Research, 14:217-231) version 97-0. The individual sequences of the pZero1 derivatives were assembled to a continuous contig. The computer-assisted coding region analysis were prepared with the XNIP program (Staden, 1986, Nucleic Acids Research, 14:217-231). Further analyses were carried out with the “BLAST search program” (Altschul et al., 1997, Nucleic Acids Research, 25:3389-3402), against the non-redundant databank of the “National Center for Biotechnology Information” (NCBI, Bethesda, Md., USA).

The resulting nucleotide sequence is shown in SEQ ID NO:4. Analysis of the nucleotide sequence showed an open reading frame of 1737 base pairs, which was called the poxB gene. The poxB gene codes for a polypeptide of 579 amino acids (SEQ ID NO:5).

### EXAMPLE 10

### Preparation of an Integration Vector for Integration Mutagenesis of the poxB Gene

From the strain ATCC 13032, chromosomal DNA was isolated by the method of Eikmanns et al. (Microbiology 140: 1817-1828 (1994)). On the basis of the sequence of the poxB gene known for C. glutamicum from Example 8, the following oligonucleotides were chosen for the polymerase chain reaction:

| | |
|---|---|
| poxBint1: | |
| 5′ TGC GAG ATG GTG AAT GGT GG 3′ | (SEQ ID NO:19) |
| poxBint2: | |
| 5′ GCA TGA GGC AAC GCA TTA GC 3′ | (SEQ ID NO:20) |

The primers shown were synthesized by MWG Biotech (Ebersberg, Germany) and the PCR reaction was carried out by the standard PCR method of Innis et al. (PCR protocols. A guide to methods and applications, 1990, Academic Press) with Pwo-Polymerase from Boehringer. With the aid of the polymerase chain reaction, a DNA fragment approx. 0.9 kb in size was isolated, this carrying an internal fragment of the poxB gene and being shown in SEQ ID NO:6.

The amplified DNA fragment was ligated with the TOPO TA Cloning Kit from Invitrogen Corporation (Carlsbad, Calif., USA; Catalogue Number K4500-01) in the vector pCR2.1-TOPO (Mead at al. (1991) Bio/Technology 9:657-663). The E. coli strain DH5α was then electroporated with the ligation batch (Hanahan, In: DNA cloning. A Practical Approach. Vol. I, IRL-Press, Oxford, Washington D.C., USA, 1985). Selection for plasmid-carrying cells was made by plating out the transformation batch on LB agar (Sambrook et al., Molecular cloning: a laboratory manual. 2^{nd} Ed. Cold Spring Harbor Laboratory Press, Cold Spring Harbor, N.Y., 1989), which had been supplemented with 25 mg/l kanamycin. Plasmid DNA was isolated from a transformant with the aid of the QIAprep Spin Miniprep Kit from Qiagen and checked by restriction with the restriction enzyme EcoRI and subsequent agarose gel electrophoresis (0.8%). The plasmid was called pCR2.1poxBint (FIG. 5).

Plasmid pCR2.1poxBint has been deposited in the form of the strain Escherichia coli DH5α/pCR2.1poxBint as DSM 13114 at the Deutsche Sammlung für Mikroorganismen und Zellkulturen (DSMZ=German Collection of Microorganisms and Cell Cultures, Braunschweig, Germany) in accordance with the Budapest Treaty.

### EXAMPLE 11

### Integration Mutagenesis of the poxB Gene in the Lysine Producer DSM 5715

The vector pCR2.1poxBint mentioned in Example 10 was electroporated by the electroporation method of Tauch et al. (FEMS Microbiological Letters, 123:343-347 (1994)) in Corynebacterium glutamicum DSM 5715. Strain DSM 5715 is an AEC-resistant lysine producer. The vector pCR2.1poxBint cannot replicate independently in DSM5715 and is retained in the cell only if it has integrated into the chromosome of DSM 5715. Selection of clones with pCR2.1 poxBint integrated into the chromosome was carried out by plating out the electroporation batch on LB agar (Sambrook et al., Molecular cloning: a laboratory manual. 2^{nd} Ed. Cold Spring Harbor Laboratory Press, Cold Spring Harbor, N.Y.), which had been supplemented with 15 mg/l kanamycin. For detection of the integration, the poxBint fragment was labeled with the Dig hybridization kit from Boehringer by the method of “The DIG System Users Guide for Filter Hybridization” of Boehringer Mannheim GmbH (Mannheim, Germany, 1993). Chromosomal DNA of a potential integrant was isolated by the method of Eikmanns et al. (Microbiology 140: 1817-1828 (1994)) and in each case cleaved with the restriction enzymes SalI, SacI and HindIII. The fragments formed were separated by agarose gel electrophoresis and hybridized at 68° C. with the Dig hybridization kit from Boehringer. The plasmid pCR2.1poxBint mentioned in Example 9 had been inserted into the chromosome of DSM5715 within the chromosomal poxB gene. The strain was called DSM5715::pCR2.1poxBint.

### EXAMPLE 12

### Effect of Over-Expression of the zwf Gene with Simultaneous Elimination of the poxB Gene on the Preparation of Lysine

12.1 Preparation of the Strain DSM5715::pCR2.1 poxBint/pEC-T18mob2zwf

The strain DSM5715::pCR2.1poxBint was transformed with the plasmid pEC-T18mob2zwf using the electroporation method described by Liebl et al., (FEMS Microbiology Letters, 53:299-303 (1989)). Selection of the transformants took place on LBHIS agar comprising 18.5 g/l brain-heart infusion broth, 0.5 M sorbitol, 5 g/l Bacto-tryptone, 2.5 g/l Bacto-yeast extract, 5 g/l NaCl and 18 g/l Bacto-agar, which had been supplemented with 5 mg/l tetracycline and 25 mg/l kanamycin. Incubation was carried out for 2 days at 33° C.

Plasmid DNA was isolated in each case from a transformant by conventional methods (Peters-Wendisch et al., 1998, Microbiology 144, 915-927), cleaved with the restriction endonucleases XbaI and KpnI, and the plasmid was checked by subsequent agarose gel electrophoresis. The strain obtained in this way was called DSM5715:pCR2.1poxBint/pEC-T 18mob2zwf.

12.2 Preparation of L-Lysine

The C. glutamicum strain DSM5715::pCR2.1poxBint/pEC-T18mob2zwf obtained in Example 12.1 was cultured in a nutrient medium suitable for the production of lysine and the lysine content in the culture supernatant was determined. For this, the strain was first incubated on an agar plate with the corresponding antibiotic (brain-heart agar with tetracycline (5 mg/l) and kanamycin (25 mg/l)) for 24 hours at 33° C. The comparison strains were supplemented according to their resistance to antibiotics. Starting from this agar plate culture, a preculture was seeded (10 ml medium in a 100 ml conical flask). The complete medium Cg III was used as the medium for the preculture.

### Medium Cg III

| | |
|---|---|
| NaCl | 2.5 g/l |
| Bacto-Peptone | 10 g/l |
| Bacto-Yeast extract | 10 g/l |
| Glucose (autoclaved separately) | 2% (w/v) |

The pH was brought to pH 7.4

Tetracycline (5 mg/l) and kanamycin (25 mg/l) were added to this. The preculture was incubated for 16 hours at 33° C. at 240 rpm on a shaking machine. A main culture was seeded from this preculture such that the initial OD (660 nm) of the main culture was 0.1. Medium MM was used for the main culture.

### Medium MM

| | |
|---|---|
| CSL (corn steep liquor) | 5 g/l |
| MOPS (morpholinopropanesulfonic acid) | 20 g/l |
| Glucose (autoclaved separately) | 58 g/l |
| (NH₄)₂SO₄ | 25 g/l |
| KH₂PO₄ | 0.1 g/l |
| MgSO₄ * 7 H₂O | 1.0 g/l |
| CaCl₂ * 2 H₂O | 10 mg/l |
| FeSO₄ * 7 H₂O | 10 mg/l |
| MnSO₄ * H₂O | 5.0 mg/l |
| Biotin (sterile-filtered) | 0.3 mg/l |
| Thiamine * HCl (sterile-filtered) | 0.2 mg/l |
| L-Leucine (sterile-filtered) | 0.1 g/l |
| CaCO₃ | 25 g/l |

The CSL, MOPS and the salt solution were brought to pH 7 with aqueous ammonia and autoclaved. The sterile substrate and vitamin solutions were then added, as well as the CaCO₃ autoclaved in the dry state. Culturing is carried out in a 10 ml volume in a 100 ml conical flask with baffles. Tetracycline (5 mg/l) and kanamycin (25 mg/l) were added. Culturing was carried out at 33° C. and 80% atmospheric humidity.

After 72 hours, the OD was determined at a measurement wavelength of 660 nm with a Biomek 1000 (Beckmann Instruments GmbH, Munich). The amount of lysine formed was determined with an amino acid analyzer from Eppendorf-BioTronik (Hamburg, Germany) by ion exchange chromatography and post-column derivation with ninhydrin detection. The result of the experiment is shown in Table 4.

TABLE 4

| | OD | L-Lysine HCl |
|---|---|---|
| Strain | (660 nm) | g/l |
| DSM5715 | 10.8 | 16.0 |
| DSM5715/pEC-T18mob2zwf | 8.3 | 17.1 |
| DSM5715::pCR2.1poxBint | 7.1 | 16.7 |
| DSM5715::pCR2.1poxBint/ | 7.8 | 17.7 |
| pEC-Tmob2zwf | | |

1-16. (canceled)

17. A protein comprising the amino acid sequence of SEQ ID NO:10, wherein said protein has glucose 6-phosphate dehydrogenase enzymatic activity.

18. The protein of claim 17, wherein said protein consists of the amino acid sequence of SEQ ID NO:10.

19. A polynucleotide consisting essentially of a nucleotide sequence encoding the protein of claim 17.

20. A polynucleotide consisting of a nucleotide sequence encoding the protein of claim 17.

21. A polynucleotide consisting of a nucleotide sequence encoding the protein of claim 18.

22. A polynucleotide consisting essentially of the sequence of nucleotides 538-2079 of SEQ ID NO:9.

23. A zwf gene consisting essentially of the nucleotide sequence of SEQ ID NO:9.

24. A vector comprising a promoter and including a region with a sequence corresponding to the nucleotide sequence of any one of claims 19-23.

25. A bacterium transformed with the vector of claim 24.

26. The bacterium of claim 25, wherein said bacterium is of the species Corynebacterium glutamicum.

27. A process for producing L-lysine, comprising:
a) fermenting the bacterium of claim 25 in a culture medium;
b) allowing L-lysine to concentrate in either said culture medium or bacterium of step a); and
c) collecting the L-lysine concentrated in step b).

28. The process of claim 27, wherein, in addition to having been transformed with the vector of claim 24, said bacterium has been modified by integration mutagenesis so that the poxB gene in said bacterium has been disrupted.

29. The process of claim 28, wherein said integration mutagenesis of said poxB gene is accomplished by transfecting said bacterium with the plasmid pCR2.1 poxBint, deposited as DSM 13114.

30. The process of claim 27, further comprising isolating said L-lysine from either said culture medium or said bacterium collected in step c).

31. A process for producing an amino acid selected from the group consisting of:
L-threonine; L-isoleucine; and L-tryptophan, comprising:
a) fermenting the bacterium of claim 25 in a culture medium;
b) allowing said amino acid to concentrate in either said culture medium or said bacterium of step a; and
c) collecting said amino acid concentrated in step b).

32. The process of claim 31, wherein, in addition to having been transformed with the vector of claim 24, said bacterium has been modified by integration mutagenesis so that the poxB gene in said bacterium has been disrupted.

33. The process of claim 32, wherein said integration mutagenesis of said poxB gene is accomplished by transforming said bacterium with the plasmid pCR2.1poxBint, deposited as DSM 13114.

34. The process of claim 31, further comprising isolating said amino acid from either said culture medium or said bacterium collected in step c).

35. A process for the production of L-lysine, comprising:
a) fermenting a bacterium producing said L-lysine in a culture medium, wherein said bacterium is of the species Corynebacterium glutamicum and wherein the zwf gene encoding the polypeptide of SEQ ID NO:10 is overexpressed in said bacterium by increasing the copy number of said zwf gene or by operably linking a promoter to said zwf gene;
b) concentrating L-lysine in either said culture medium or said bacterium of step a); and
c) collecting the L-lysine concentrated in step b).

36. The process of claim 35, wherein said zwf gene comprises the nucleotide sequence of SEQ ID NO:9.

37. The process of claim 35, wherein, in addition to the overexpression of said zwf gene, the poxB gene in said bacterium has been disrupted by integration mutagenesis.

38. The process of claim 37, wherein said integration mutagenesis of said poxB gene is accomplished by transforming said bacterium with the plasmid pCR2.1 poxBint, deposited as DSM 13114.

39. The process of claim 35, further comprising isolating said L-lysine from either said culture medium or said bacterium collected in step c).

40. A process for the production of an amino acid selected from the group consisting of: L-threonine; L-isoleucine; and L-tryptophan, comprising:
a) fermenting a bacterium producing said L-amino acid in a culture medium, wherein said bacterium is of the species Corynebacterium glutamicum; and the zwf gene encoding the polypeptide of SEQ ID NO:10 is overexpressed in said bacterium by increasing the copy number of said zwf gene or by operably linking a promoter to said zwf gene;
b) allowing said amino acid to concentrate in either said culture medium or said bacterium of step a); and
c) collecting said amino acid concentrated in step b).

41. The process of claim 40, wherein said zwf gene comprises the nucleotide sequence of SEQ ID NO:9.

42. The process of claim 40, wherein, in addition to said zwf gene being overexpressed, the poxB gene in said bacterium has been disrupted by integration mutagenesis.

43. The process of claim 42, wherein said integration mutagenesis of said poxB gene is accomplished by transforming said bacterium with the plasmid pCR2.1 poxBint, deposited as DSM 13114.

44. The process of claim 40, further comprising isolating said amino acid from either said culture medium or said bacterium collected in step c).
